(19) United States
(12) Patent Application Publication
(10) Pub. No.: US 2020/0165574 A1
(43) Pub. Date: May 28, 2020

(54) METHODS OF PREPARING NAÏVE HUMAN PLURIPOTENT STEM CELLS

(71) Applicants: Jakub Tolar, Minneapolis, MN (US); Kirk Robert Twaroski, Minneapolis, MN (US); Emily Faith Ward, Minneapolis, MN (US); James Dutton, Minneapolis, MN (US); REGENTS OF THE UNIVERSITY OF MINNESOTA, Minneapolis, MN (US)

(72) Inventors: Jakub Tolar, Minneapolis, MN (US); Kirk Robert Twaroski, Minneapolis, MN (US); Emily Faith Ward, Minneapolis, MN (US); James Dutton, Minneapolis, MN (US)

(73) Assignee: REGENTS OF THE UNIVERSITY OF MINNESOTA, Minneapolis, MN (US)

(21) Appl. No.: 16/612,900

(22) PCT Filed: May 11, 2018
(86) PCT No.: PCT/US18/32315

§ 371 Date: Nov. 12, 2019

Related U.S. Application Data
(60) Provisional application No. 62/505,273, filed on May 12, 2017.

(51) Int. Cl. C12N 5/074

(52) U.S. Cl. CPC C12N 5/0696; C12N 2533/50; C12N 2501/33; C12N 2500/98; C12N 2501/115; C12N 2501/15; C12N 2501/998; C12N 2501/113

(57) ABSTRACT

Methods of preparing naive human pluripotent stem cells are described. The methods include the use of xeno-free media and do not include the use of feeder cells.

### CONTINUING APPLICATION DATA

This application claims the benefit of U.S. Provisional Application Ser. No. 62/505,273, filed May 12, 2017, which is incorporated by reference herein.

### SEQUENCE LISTING

This application contains a Sequence Listing electronically submitted to the United States Patent and Trademark Office via EFS-Web as an ASCII text file entitled “110-05800201_ST25.txt” having a size of 4 kilobytes and created on May 11, 2018. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY OF THE INVENTION

This disclosure describes methods for preparing a naïve human pluripotent stem cell. In one aspect, the method includes providing a human pluripotent stem cell (HPSC) and culturing the HPSC in the absence of feeder cells and in the presence of vitronectin.

In some embodiments, providing a HPSC includes providing a primed HPSC.

In some embodiments, the vitronectin includes full-length vitronectin. In some embodiments, the vitronectin includes vitronectin coated on a surface.

In some embodiments, culturing the HPSC in the absence of feeder cells includes culturing the HPSC in a medium that includes insulin, fibroblast growth factor (FGF), transforming growth factor beta (TGFβ), and/or Activin.

In some embodiments, the method further includes culturing the HPSC in the absence of FGF and TGFβ. The cells may be cultured under hypoxic conditions.

In some embodiments, the method further includes culturing the HPSC in a medium including insulin, FGF, TGFβ, and/or Activin prior to culturing the HPSC in the presence of vitronectin.

In some embodiments, culturing the HPSC includes culturing the HPSC in a xeno-free medium.

In another aspect, the method includes providing a HPSC; culturing the HPSC in the absence of feeder cells and in the presence of vitronectin coated on a surface in a xeno-free medium including insulin, FGF, TGFβ, and/or Activin; and then culturing the HPSC under hypoxic conditions in a xeno-free medium in the absence of FGF and TGFβ. The vitronectin may include full-length vitronectin. The HPSC may be a primed HPSC.

In some embodiments, the method may further include culturing the HPSC in a xeno-free medium including insulin, fibroblast growth factor (FGF), transforming growth factor beta (TGFβ), and/or Activin prior to culturing the primed HPSC in the presence of vitronectin.

In some embodiments, the naïve HPSC exhibits a normal karyotype. In some embodiments, the naïve HPSC does not differentiate in the presence of an ERK inhibitor.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows that naïve pluripotent stem cells derived on vitronectin according to the methods described in Example 1 are distinct from primed pluripotent stem cells. (A) Morphology of a primed HPSC colony. (B) Morphology of a transitioned naïve HPSC colony (Passage 10). ERK inhibitor PD0325901 induces differentiation in primed HPSC (C), as evidenced by morphological changes, but not in naïve HPSC (D). Expression of NANOG, a transcription factor involved with self-renewal of undifferentiated embryonic stem cells, in primed (E) and transitioned (F) naïve HPSCs. Expression of stage-specific embryonic antigen-3 (SSEA3), a marker of stem cell-like characteristics, in primed (G) and transitioned (H) naïve HPSCs. Expression of stage-specific embryonic antigen-4 (SSEA4), a marker of stem cell-like characteristics, in primed (I) and transitioned (J) naïve HPSCs.

FIG. 2A and FIG. 2(B-J) show naïve pluripotent stem cells derived on vitronectin have a normal karyotype and pluripotent differentiation potential. FIG. 2A shows a representative karyotype of naïve HPSCs after 10 passages in RSet media. FIG. 2(B-J) shows exemplary differentiated naïve cells express the endoderm markers FOXA2 (B) and SOX17 (C), mesoderm markers CXCR4 (E) and Brachyury (F), and the ectoderm markers PAX6 (H) and Nestin (I). Corresponding DAPI staining is shown for the endoderm markers (D), the mesoderm markers (G), and the ectoderm markers (J).

FIG. 3 shows expression of pluripotent markers in primed and naïve HPSCs. Expression of the pluripotency markers CDH1 and OCT4 is changed less than two-fold in naïve HPSCs compared to primed HPSCs while expression of NANOG is increased in naïve HPSCs compared to primed HPSCs. P18: primed HPSCs passage 18; N7: naïve HPSCs passage 7; N10: naïve HPSCs passage 10; N12: naïve HPSCs passage 12.

FIG. 4 shows exemplary morphology of naïve pluripotent stem cells derived in feeder-free conditions. (A) Morphology of a primed HPSC colony cultured on vitronectin. Colony morphology was followed during transition in RSet media after passage 1 (B), passage 7 (C) and passage 10 (D). (E) Morphology of a primed HPSC colony cultured on Matrigel. Colony morphology was followed during transition in RSet media after passage 1 (F), passage 7 (G), and passage 10 (H).

### DETAILED DESCRIPTION OF ILLUSTRATIVE EMBODIMENTS

This disclosure describes methods for the derivation of a naïve human pluripotent stem cell (HPSC) from a primed HPSC using defined conditions.

Human pluripotent stem cells (HPSCs) cultured in conditions that maintain pluripotency via fibroblast growth factor (FGF) and transforming growth factor beta (TGFβ) signaling are described as being in a primed state. These cells have been shown to exhibit characteristics more closely related to mouse epiblast-derived stem cells than to so-called naïve mouse pluripotent stem cells said to possess a more ground state pluripotency that mimics the early mouse embryo inner cell mass (Tesar et al. (2007) Nature, 448: 196-199; Hanna et al. (2010) PNAS, 107: 9222-9227).

At the time of the invention, culture conditions favorable for generation of naïve HPSCs from primed HPSCs required the use of mouse embryonic fibroblasts as a feeder layer or a mixture of truncated vitronectin mixed with gelatin to support the transition from primed HPSCs to a naïve HPSCs (Theunissen et al. (2014) Cell Stem Cell, 15: 471-487; Gafni et al. (2013) Nature, 504: 282-286). This disclosure describes a protocol for producing naïve HPSCs from primed HPSCs in defined, xeno-free conditions. The methods described herein further describe maintenance of naïve HPSCs in defined, xeno-free conditions. These methods are expected to allow stem cell researchers to enhance the study and clinical translation of naïve HPSCs.

In one aspect, this disclosure describes a method for preparing a naïve human pluripotent stem cell. The method includes providing a HPSC and culturing the HPSC in the absence of feeder cells and in the presence of vitronectin. In some embodiments, culturing the HPSC in the absence of feeder cells preferably includes culturing the HPSC in the absence of gelatin.

As used herein, a feeder cell is a cell on which stem cells, particularly a HPSC, may be plated and/or which provide a milieu conducive to the growth and maintenance of the stem cells in a pluripotent state. In some embodiments, culturing the HPSC in the absence of feeder cells includes culturing the HPSC in the absence of a conditioned medium. A conditioned medium is a medium taken from a culture of a feeder cell to maintain the HPSC in a pluripotent state without direct contact with the feeder cells.

In some embodiments, the HPSC provided is a primed HPSC. As used herein, a “primed HPSC” is a cell characterized by a flattened morphology (in a colony of cells), intolerance to passaging as single cells, and a dependence on bFGF and TGFβ/Activin signaling rather than LIF/Stat3 (Hanna et al. (2010) PNAS, 107: 9222-9227).

As used herein, a “naïve HPSC” is a cell that can be cloned with high efficiency, can (in a colony of cells) grow in a packed dome colony, and is stabilized by LIF/Stat3 and destabilized by bFGF and TGFβ/Activin signaling. In some embodiments, a “naïve HPSC” is characterized by having a tightly packed cell morphology that (in a colony of cells) forms a rounded, three-dimensional colony with distinct phase bright edges. In some embodiments, a “naïve HPSC” can be passaged routinely using Tryp1E mediated single cell dissociation.

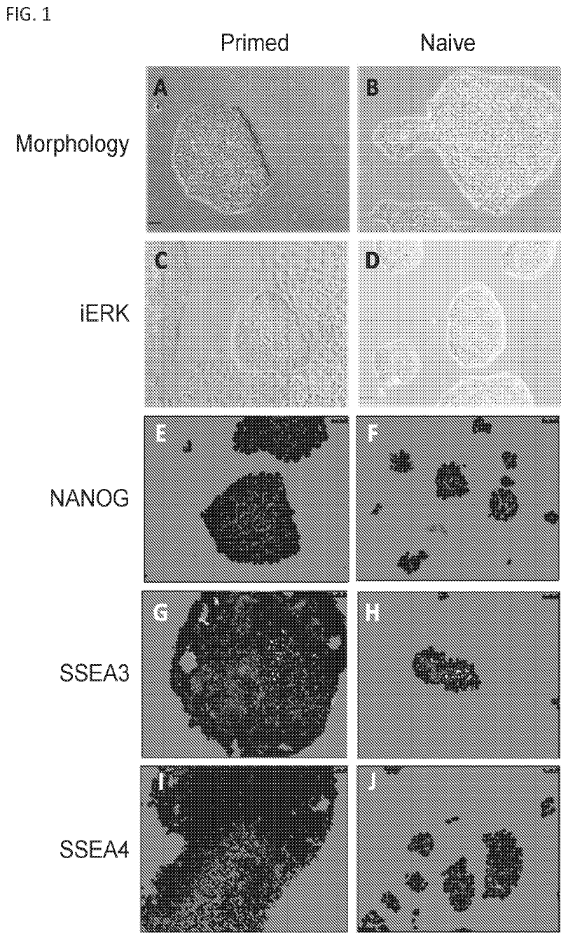

For example, the representative images in FIG. 1 of primed HPSCs show flattened cell colonies that are made up of numerous flattened individual cells; in contrast, the representative images in FIG. 1 of naïve HPSCs show more rounded cells that contribute to a more rounded, dome-like colony.

The primed HPSC can include any suitable primed HPSC. In some embodiments, the primed HPSC is a primed human induced pluripotent stem cell (hiPSC). In some embodiments, a hiPSC can include a cell from the PCBC16ipS cell line (Ye et al. (2013) PLoS ONE 8 (1):e53764).

In some embodiments the vitronectin includes a full-length vitronectin. In some embodiments the vitronectin is a human vitronectin. In some embodiments, the vitronectin may include the amino acid sequence of SEQ ID NO:1. In some embodiments, the vitronectin may be recombinant. In some embodiments, the vitronectin is full-length vitronectin from PeproTech, Rocky Hill, N.J.

In some embodiments the vitronectin may preferably be coated on a surface. The surface may include a cell culture surface including, for example, a plate. In some embodiments, the vitronectin may be coated at a concentration of at least 1 microgram per milliliter (μg/mL), at least 2 μg/mL, at least 3 μg/mL, at least 4 μg/mL, or at least 5 μg/mL. In some embodiments, the vitronectin may be coated at a concentration of up to 2 μg/mL, up to 3 μg/mL, up to 4 μg/mL, up to 5 μg/mL, up to 6 μg/mL, up to 8 μg/mL, or up to 10 μg/mL. For example, in some embodiments, a 12-well tissue culture treated plate may preferably be coated with 5 μg/mL full-length vitronectin.

Without wishing to be bound by theory, the culture matrix including vitronectin may be the limiting factor defining a successful transition to a naïve pluripotent state in the feeder-free conditions described herein.

In some embodiments, culturing the HPSC in the absence of feeder cells may include culturing the HPSC in a medium including insulin, FGF, TGFβ, and/or Activin A. In some embodiments, the medium may preferably include mTeSR1 medium (STEMCELL Technologies, Vancouver, Canada). In some embodiments, the medium may include a FGF/TGFβ1/Activin A-containing media described by Gafni et al. (2013) Nature, 504: 282-286 and/or by “WIS-NHSM Human Naïve Stem Cell Platform Approaches and Protocols,” available on the world wide web at hannalabweb.weizmann.ac.il/wp-content/uploads/2015/08/Hanna-Lab-Detailed-and-Extended-WIS-NHSM-Formulations.pdf.

In some embodiments, the method further includes culturing the HPSC in the absence of FGF and TGFβ. In some embodiments, culturing the HPSC in the absence of FGF and TGFβ includes culturing the HPSC in the absence of Activin. In some embodiments, the HPSC may preferably be cultured in a medium including RSet media (STEMCELL Technologies, Vancouver, Canada). In some embodiments, the HPSC may be cultured in a medium including a FGF/TGFβ1/Activin A-free media described by Gafni et al. (2013) Nature, 504: 282-286 and/or by “WIS-NHSM Human Naïve Stem Cell Platform Approaches and Protocols,” available on the world wide web at hannalabweb.weizmann.ac.il/wp-content/uploads/2015/08/Hanna-Lab-Detailed-and-Extended-WIS-NHSM-Formulations.pdf.

For example, the HPSC may be cultured in the absence of feeder cells and in the presence of vitronectin, FGF, TGFβ, and/or Activin, and then the HPSC may be cultured in the absence of FGF and TGFβ. In some embodiments, the HPSC may be cultured in the absence of FGF and TGFβ under hypoxic conditions. As used herein, “hypoxic conditions” may be defined as conditions having an oxygen level in a range from 1% to 15%. In some embodiments, hypoxic conditions may be defined as conditions having an oxygen level of 5%.

In some embodiments, the method further includes culturing the HPSC in a medium including insulin, FGF, TGFβ, and/or Activin. In some embodiments, the medium may preferably include mTeSR1 medium (STEMCELL Technologies, Vancouver, Canada). In some embodiments, the medium may include a FGF/TGFβ1/Activin A-containing media described by Gafni et al. (2013) Nature, 504: 282-286 and/or by “WIS-NHSM Human Naïve Stem Cell Platform Approaches and Protocols,” available on the world wide web at hannalabweb.weizmann.ac.il/wp-content/uploads/2015/08/Hanna-Lab-Detailed-and-Extended-WIS-NHSM-Formulations.pdf. In some embodiments, the HPSC may be cultured in a medium including insulin, FGF, TGFβ, and/or Activin prior to culturing the HPSC in the absence of feeder cells and in the presence of vitronectin.

In some embodiments, culturing the HPSC includes culturing the HPSC in a xeno-free medium during part of the culturing or during each part of the culturing. As used herein, a “xeno-free” medium is a medium that does not include a component derived from a different organism than the cell being cultured. In some embodiments, a “xeno-free” medium but may contain a component derived from the same organism as the cell being cultured. In some embodiments, a “xeno-free” medium is gelatin-free.

The methods described herein are intended to produce a naïve HPSC. In some embodiments, the naïve HPSC preferably exhibits a normal karyotype. In some embodiments, the naïve HPSC preferably does not differentiate in the presence of an ERK inhibitor including, for example, PD0325901.

In some embodiments, the naïve HPSC expresses markers characteristic of HPSCs including, for example, CDH1 and/or OCT4. In some embodiments, the naïve HPSC has a level of expression of CDH1 and/or OCT4 within two-fold of the gene expression of a primed HPSC. In some embodiments, the naïve HPSC has increased NANOG protein expression or NANOG gene expression relative to the protein or gene expression of a primed HPSC. In some embodiments, the naïve HPSC have at least 1.1 fold, at least 1.3 fold, or at least 1.5 fold increased NANOG gene expression relative to the NANOG gene expression of a primed HPSC.

In some embodiments, the naïve HPSC exhibits an ability to differentiate into three germ layer derivatives. In some embodiments, differentiation into three germ layer derivatives can be determined in vivo including, for example, by injection of cells into mice followed by pathological examination of hte presence of tissue from the three germ layers. In some embodiments, differentiation into three germ layer derivatives can be determined in vitro. In some embodiments, differentiation into three germ layers can be measured using STEMdiff Trilineage Differentiation Kit (05230, STEMCELL Technologies, Vancouver, Canada).

In some embodiments, the naïve HPSC can be passaged. In some embodiments, the naïve HPSC can be stably maintained after being passaged at least 10 times, at least 15 times, at least 20 times, at least 25 times, or at least 30 times. In some embodiments, the naïve HPSC can be passaged using a TrypLE reagent (Thermo Fisher Scientific, Waltham, Mass.)-mediated single cell dissociation.

The present invention is illustrated by the following examples. It is to be understood that the particular examples, materials, amounts, and procedures are to be interpreted broadly in accordance with the scope and spirit of the invention as set forth herein.

### EXAMPLE

### Example 1

This Example describes an exemplary protocol for transitioning primed HPSCs to a naïve state using commercial RSet media (STEMCELL Technologies, Vancouver, Canada) and xeno-free recombinant full-length vitronectin.

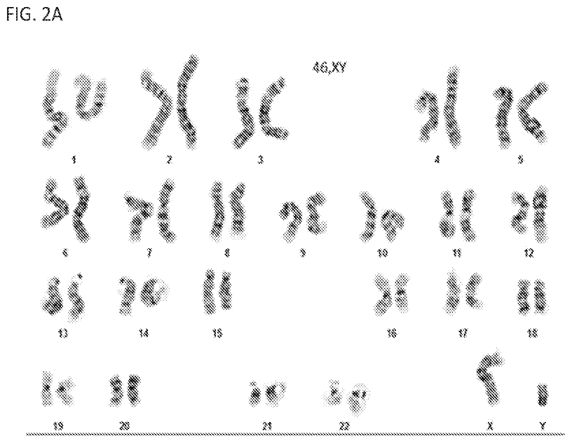

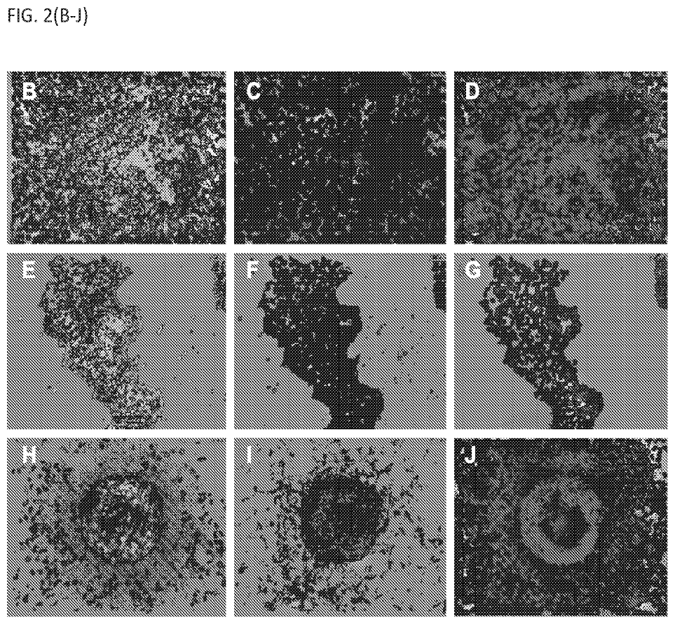

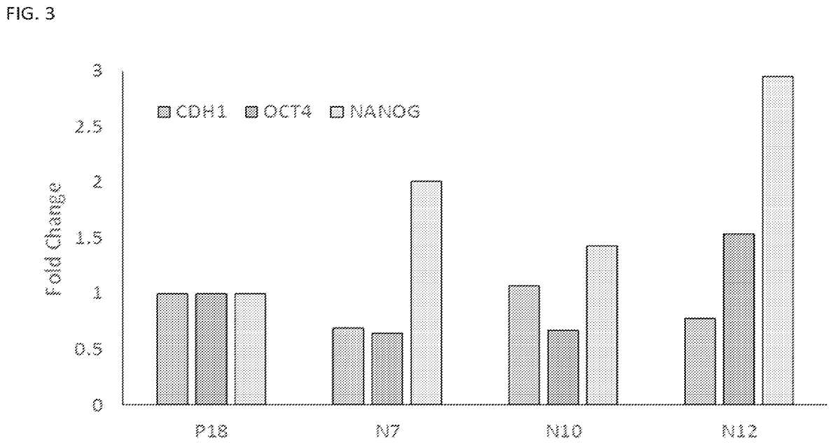

Primed HPSCs maintained in defined conditions on a recombinant vitronectin substrate have a stereotypical morphology characterized by small cells with a large nuclear:cytoplasmic ratio that form flat monolayer colonies (FIG. 1A). After transition to the naïve state, the HPSCs assumed a tightly packed cell morphology forming rounded, three-dimensional colonies with distinct phase bright edges (FIG. 1B). The naïve cells can be passaged using TrypLE-mediated single cell dissociation. In contrast to other published protocols for feeder-free naïve HPSC derivation, the cells transitioned to the naïve state using the protocol of this Example exhibit a normal karyotype (FIG. 2A). When cultured in media including 10 nanomolar (nM) of an ERK inhibitor (PD0325901), primed HPSCs were not able to maintain pluripotency and displayed immediate differentiation (FIG. 1C). However, ERK signaling has been reported as dispensable for naïve HPSCs to maintain a pluripotent state, and naïve cells transitioned from the primed state maintain their undifferentiated state in RSet media supplemented with an additional 10 nM PD325901 (FIG. 1D). The transitioned naïve HPSCs maintained expression of markers characteristic of HPSCs and could differentiate into three germ layer derivatives in vitro (FIG. 2). Expression of the pluripotent markers CDH1 and OCT4 were changed less than 2-fold in naïve HPSCs from primed HPSCs while expression of NANOG was increased (FIG. 3). The cells were stably maintained in the naïve state culture conditions for more than 30 passages.

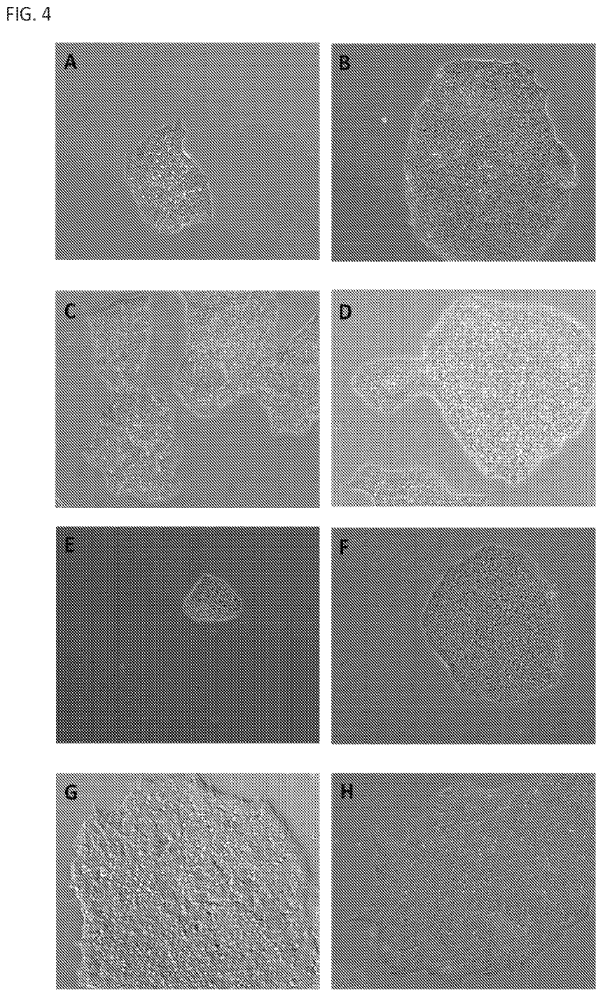

The use of a full-length recombinant vitronectin in the manner described herein during transition supports the successful transition between pluripotent states; whereas, the use of Matrigel did not support a successful transition (FIG. 4). Although the HPSCs transitioned to the naïve state on Matrigel had many of the hallmarks of naïve pluripotent stem cells, karyotype analysis revealed clonal abnormalities in all transitioned cell lines.

The derivation of naïve HPSCs from primed HPSCs on vitronectin was repeated with multiple primed HPSC lines from various genetic backgrounds.

### Materials and Methods

### Feeder Free Naïve Pluripotent Stem Cell Derivation

Primed induced pluripotent stem cell (PCBC16iPS) (Ye et al. (2013) PLoS ONE 8 (1):e53764) to be used for naïve cell derivation were cultured for at least two passages in mTeSR1 medium (STEMCELL Technologies, Vancouver, Canada) in either normoxic (20% O₂, 5% CO₂) or hypoxic conditions (5% O₂, 5% CO₂). Primed iPSCs were treated with Gentle Cell Dissociation Reagent (STEMCELL Technologies, Vancouver, Canada) for 5 minutes at room temperature and removed from the plate with a 5 milliliter (mL) pipette in 1 mL TeSR1 medium. Aggregates of 250 primed iPSCs were plated in 1 mL mTeSR1 (STEMCELL Technologies, Vancouver, Canada) per well of a 12-well tissue culture treated plate (Corning, Inc., Corning, N.Y.) coated with 5 micrograms per milliliter (μg/mL) full-length vitronectin (PeproTech, Rocky Hill, N.J.) (Parr et al. (2016) Methods in Molecular Biology 1357:221-9). The plate had been previously coated with vitronectin at 37° C. for 2 hours per manufacturer's protocol. The plated cells were incubated overnight at 37° C., to allow adherence to the vitronectin coated plate. The cells may be incubated in either normoxic or hypoxic conditions, and typically were incubated under the same conditions used to maintain the primed HPSC culture. The next day, cells were transferred to hypoxic culture conditions and switched to RSeT medium (STEMCELL Technologies, Vancouver, Canada). Cells were monitored for compaction and defined colony edges. When naïve iPSC colonies reached 250 millimeters (mm) in diameter, the cells in the well were passaged. For passaging, cells were washed one time with phosphate buffered saline (PBS) without Ca²⁺ or Mg²⁺, followed by addition of 250 μL TrypLE Express (Thermo Fisher Scientific, Waltham, Mass.) for 3 minutes at 37° C. in hypoxic conditions. TrypLE was neutralized with 750 μL RSet supplemented with 10 μM ROCK inhibitor Y-27632 (BD Biosciences, San Jose, Calif.). Cells were agitated by pipetting 3 times using a P1000 pipet tip to achieve 10-12 cell clumps. After collection, cells were spun at 300×g for 5 minutes. Cells were resuspended in 120 μL of RSeT medium supplemented with 10 μM Y-27632 by pipetting 6 times with a P200 pipet tip to break cells into clumps of 2-3 cells. Cells were plated in dilutions of 1:3, 1:4, 1:8, and 1:10 to ensure optimal plating density.

### ERK Inhibitor Assay

One day after passaging, 10 nM ERK inhibitor PD0325901 (PZ0162, Sigma-Aldrich, St. Louis, Mo.) was added to naïve or primed PSCs. Cells were imaged each day for 4 days to follow morphology changes.

### Immunocytochemistry

Cultured cells were fixed in 4% paraformaldehyde (PFA) for 15 minutes at room temperature (20° C.-25° C.) and then made permeable with 0.2% Triton X-100 in PBS for 30 minutes. Cells were blocked with 3% bovine serum albumin (BSA) in phosphate buffered saline (PBS) for 2 hours, then incubated overnight at 4° C. with primary antibodies diluted in 3% BSA. The antibodies used were SSEA-3 (1 μg/mL; MAB4303, Millipore, Billerica, Mass.), and SSEA-4 (1 μg/mL; MAB4304, Millipore, Billerica, MA). Cells were washed in PBS 3 times (for 5 minutes each wash) and incubated with 0.5 μg/mL respective secondary antibody (A21042, Alexa Fluor anti-goat 488; A21432, Alexa Fluor anti-goat 555; A21434, Alexa Fluor anti-rat 555; A31572, Alexa Fluor anti-rabbit 555; A11001, Alexa Fluor anti-goat 488, all from Life Technologies, Thermo Fisher Scientific, Waltham, Mass.) for 1 hour at room temperature and washed with PBS. The cells were stained for 10 minutes at room temperature with 4,6-diamidino-2-phenylindole (DAPI, 1 μg/mL; Invitrogen Corporation, Carlsbad, Calif.) diluted in PBS. Images were processed using Adobe Photoshop to optimize brightness and contrast, with all control and experimental images being treated identically.

To detect NANOG expression, wells were fixed for 10 minutes at room temperature using formalin. The cells were permeabilized with mixture of 1% BSA and 0.2% TritonX1000 in PBS for 10 minutes. The cells were blocked with Block (1% BSA+0.1% Tween) for 30 minutes at room temperature. The cells were incubated overnight at 4° C. with the primary antibody against NANOG (5 μg/mL; AF1997, R&D Systems, Minneapolis, Minn.) diluted in Block. The primary antibody was washed off using Block, and the secondary antibody (0.5 μg/mL, A21432, Alexa Fluor anti-goat 555) was added for 1 hour at room temperature. The well was washed 3 times with PBS, and DAPI (1 μg/mL; Invitrogen Corporation, Carlsbad, Calif.) diluted in PBS was added for 5 minutes at room temperature. The imaging was performed as described above.

### Pluripotency Assay

The differentiation potential of naïve HPSCs into each of the three germ layers was performed using STEMdiff Trilineage Differentiation Kit (05230, STEMCELL Technologies, Vancouver, Canada). Briefly, cells were plated onto Matrigel (Corning, Inc., Corning, N.Y.) and treated with STEMdiff Trilineage Endoderm Medium or STEMdiff Trilineage Mesoderm Medium for 5 days or STEMdiff Trilineage Ectoderm Medium for 7 days. Cells were then fixed, stained, and imaged as described above.

### Karyotype

Naïve HPSCs were examined by high-resolution G banding after 10 passages in RSet medium.

### RT-qPCR

RNA was isolated from primed HPSCs at passage 18 and naïve HPSCs at passage 7, passage 10, and passage 12 using the RNeasy RNA Isolation Kit (Qiagen Company, Hilden, Germany), and cDNA was prepared using Superscript IV (Invitrogen Corporation, Carlsbad, Calif.). RT-qPCR was run on the Mastercycler epgradient S (Eppendorf, Hamburg, Germany) and analyzed using ep realplex software (Eppendorf, Hamburg, Germany). PrimeTime assays for CDH1 (Hs.PT.58.3324071), POU5F1 (Hs.PT.58.14648152.g), NANOG (Hs.PT.58.21480849), and GAPDH (Hs.PT.39a.22214836) were obtained from Integrated DNA Technologies (Coralville, Iowa).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for preparing a naïve human pluripotent stem cell, the method comprising:
providing a human pluripotent stem cell (HPSC); and
culturing the HPSC in the absence of feeder cells and in the presence of vitronectin;
thereby producing a naïve HPSC.

2. The method of claim 1, wherein providing a HPSC comprises providing a primed HPSC.

3. The method of claim 1, wherein the vitronectin comprises full-length vitronectin.

4. The method of claim 1, wherein the vitronectin comprises vitronectin coated on a surface.

5. The method of claim 1, wherein culturing the HPSC in the absence of feeder cells comprises culturing the HPSC in a medium comprising at least one of insulin, fibroblast growth factor (FGF), transforming growth factor beta (TGFβ), and Activin.

6. The method of claim 1, the method further comprising culturing the HPSC in the absence of fibroblast growth factor (FGF) and transforming growth factor beta (TGFβ).

7. The method of claim 6, wherein the cells are cultured under hypoxic conditions.

8. The method of claim 1, the method further comprising culturing the HPSC in a medium comprising at least one of insulin, fibroblast growth factor (FGF), transforming growth factor beta (TGFβ), and Activin prior to culturing the HPSC in the presence of vitronectin.

9. The method of claim 1, wherein culturing the HPSC comprises culturing the HPSC in a xeno-free medium.

10. A method for preparing a naïve human pluripotent stem cell, the method comprising:
providing a human pluripotent stem cell (HPSC);
culturing the primed HPSC in the absence of feeder cells and in the presence of vitronectin coated on a surface, wherein the vitronectin comprises full-length vitronectin, and wherein the HPSC are cultured in a xeno-free medium comprising at least one of insulin, fibroblast growth factor (FGF), transforming growth factor beta (TGFβ), and Activin; and
then culturing the HPSC in a xeno-free medium in the absence of fibroblast growth factor (FGF) and transforming growth factor beta (TGFβ), wherein the cells are cultured under hypoxic conditions;
thereby producing a naïve HPSC.

11. The method of claim 10, the method further comprising culturing the HPSC in a xeno-free medium comprising at least one of insulin, fibroblast growth factor (FGF), transforming growth factor beta (TGFβ), and Activin prior to culturing the primed HPSC in the presence of vitronectin.

12. The method of claim 10, wherein providing an HPSC comprises providing a primed HPSC.

13. The method of claim 1, wherein the naïve HPSC exhibits a normal karyotype.

14. The method of claim 1, wherein the naïve HPSC does not differentiate in the presence of an ERK inhibitor.

15. The method of claim 10, wherein the naïve HPSC exhibits a normal karyotype.

16. The method of claim 10, wherein the naïve HPSC does not differentiate in the presence of an ERK inhibitor.


SEQUENCE LISTING (1 sequence)

SEQ ID NO: 1 - protein, 459 aa, Homo Sapiens
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL